(19) United States
(12) Patent Application Publication
(10) Pub. No.: US 2022/0249288 A1
(43) Pub. Date: Aug. 11, 2022

(54) FIXED DEPTH TREPHINE

(71) Applicant: Surgistar, Inc., Vista, CA (US)

(72) Inventors: Neil CHAUDHARY, Vista, CA (US); Jonathan WOODWARD, Vista, CA (US)

(21) Appl. No.: 17/581,328

(22) Filed: Jan. 21, 2022

Related U.S. Application Data
(60) Provisional application No. 63/141,244, filed on Jan. 25, 2021.

(51) Int. Cl. A61F 9/013; A61F 2/14; A61B 17/3205

(52) U.S. Cl. CPC A61F 9/0133; A61B 17/32053; A61F 2/142

(57) ABSTRACT

Provided herein is a single piece fixed depth trephine for use in ophthalmic surgeries and other surgical applications. Related methods are also described.

### CROSS REFERENCE TO RELATED APPLICATIONS

This application claims priority to U.S. Provisional Application No. 63/141,244, filed Jan. 25, 2021, which is hereby incorporated by reference in its entirety and for all purposes.

### BACKGROUND

Fixed depth trephines are used in many applications, including corneal surgery where a cut must be created to a known depth to form a partial thickness cut. These partial thickness cuts can be used for deep anterior lamellar keratoplasty (DALK), Descemet membrane endothelial keratoplasty (DMEK), and other partial graft surgeries.

The current manufacturing process for fixed depth trephines requires the use of at least three distinct parts: a cutting blade (trephine), an outer housing, and an inner reticle. These parts are then glued or press-fit to form a final configuration. This can increase production costs and the complexity of the configuration. There is a need for a single-piece fixed-depth trephine that decreases production cost and complexity.

### BRIEF SUMMARY

Described and illustrated herein is a one-piece fixed depth trephine that addresses this and other problems in the art.

In an aspect, a surgical instrument is provided. The surgical instrument includes a cutting blade; an outer housing having an outer diameter; and an inner housing having an inner diameter and an inner diameter shelf, wherein the inner diameter shelf is configured as a depth fixation apparatus.

In some embodiments, the cutting blade, the outer housing, the inner housing, and the inner diameter shelf are configured as a single piece. In other embodiments, the cutting blade, the outer housing, and the inner housing are configured as a first piece, and the inner diameter shelf is configured as a second piece insertable into the inner housing of the first piece. In some embodiments, the surgical instrument is disposable. In other embodiments, the surgical instrument is reusable. In some embodiments, the surgical instrument is a trephine.

In another, interrelated aspect, a method of forming an incision in a tissue is provided. The method includes setting a desired blade depth using a depth fixation apparatus of a surgical instrument; placing an outer housing of the surgical instrument on a surface of the tissue to be cut; and forming an incision in the tissue, the incision having a desired depth.

In some embodiments, the depth fixation apparatus of the surgical instrument comprises an inner diameter shelf. In some embodiments, the inner diameter shelf forms a contiguous part of the surgical instrument. In other embodiments, the inner diameter shelf comprises a separate piece that is coupled to the surgical instrument. In some embodiments, the surgical instrument is a trephine.

### BRIEF DESCRIPTION OF THE DRAWINGS

FIG. 1 shows front perspective and planar views of a traditional fixed depth trephine having a depth fixation apparatus on the outer diameter (OD) of a trephine blade.

FIG. 2A shows a front planar view of a fixed depth trephine having an inner diameter shelf configured to set the depth of cut.

FIG. 2B shows a side planar view of the fixed depth trephine of FIG. 2A.

### DETAILED DESCRIPTION

Described and illustrated herein is a surgical device for use in surgeries, such as ophthalmic surgeries and other procedures requiring an incision of a fixed depth. The surgical device can be a trephine, for example a fixed depth trephine. The fixed depth trephine can be a one-piece apparatus, or single piece, monolithic apparatus, which utilizes a structural configuration such as an inner diameter shelf to set a depth of a cut. In some embodiments, a separate insert may be placed within the inner diameter wherein the separate insert acts as the inner diameter shelf. In other embodiments, an inner diameter shelf may be formed as a monolithic part of the single piece apparatus.

The use of a single piece apparatus rather than a multi-piece apparatus offers the advantages of reduced production cost and complexity. Additionally, using an inner diameter depth fixation shelf rather than an outer diameter depth fixation apparatus offers improved incision depth accuracy. The depth of the incision or cut is determined by a contact radius at the points where the depth fixation apparatus contacts the patient's cornea, wherein the contact radius is an outer surface defined by a radius wherein the outer surface contacts the cornea. The contact radius on the outside of the trephine is larger, and therefore the potential for surface aberrations to adversely affect the depth determination is increased. The fixed depth trephine disclosed herein has a depth fixation apparatus located on the inner diameter of the trephine, which uses a smaller radius than the outside diameter and thereby reduces the potential for surface aberrations to adversely affect the depth determination.

Furthermore, in larger trephine sizes the outer diameter depth fixation apparatus can cross the limbus, making it considerably less accurate and potentially incorrect. By determining the depth of cut using a depth fixation apparatus positioned on the inner diameter of the trephine, the depth determination is more accurate in that the contact radius is smaller and less subject to surface aberrations. Additionally, the inner diameter depth fixation apparatus will not cross the limbus. The depth of cut being determined using the inner diameter of the trephine reduces possible errors of depth, in that the radius used to contact the eye on the inner diameter is considerably smaller than the radius being used on the outer diameter of the trephine.

The use of an inner diameter depth fixation shelf also offers the advantages of less abrasion to the cornea, less pain to the patient, less potential for infection, less surgical complications, and improved ophthalmic surgical outcomes. The portion of the cornea that contacts the fixation apparatus of a trephine is subject to abrasions from its contact with the depth fixation apparatus. Abrasions to the cornea are a source of pain, and can lead to increased healing times, inferior ophthalmic outcomes, infection, and other surgical complications. Current trephines using an outer diameter depth fixation apparatus can cause abrasions to the portion of the cornea that remains on the patient. The fixed depth trephine disclosed herein uses an inner diameter depth fixation apparatus that will not contact the portion of the cornea that remains on the patient. The fixed depth trephine's depth fixation apparatus will only contact the portion of the cornea that will be removed and discarded, thereby reducing pain to the patient, reducing the potential for infection, reducing surgical complications, and improving ophthalmic surgical outcomes.

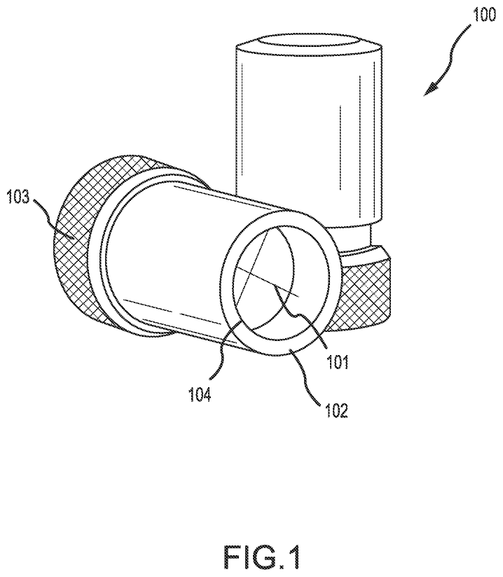

FIG. 1 illustrates a typical fixed depth trephine 100 having a depth fixation apparatus 103 on an outer diameter 102 of an actual trephine blade 101, which is positioned inside the apparatus. The fixed depth trephine 100 also has an inner diameter 104. Examples of currently available trephines include the CORONET® Guarded Corneal Trephines and the Hessburg-Barron Vacuum Trephine by JEDMED Instrument Co. Trephines may be non-vacuum, vacuum, or suction trephines.

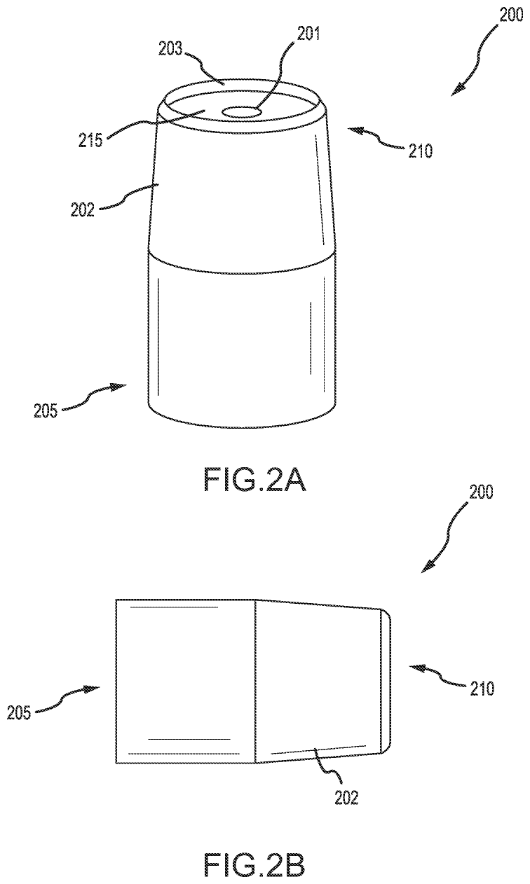

FIGS. 2A-2B illustrate a fixed depth trephine 200 configured as a single piece apparatus such as a monolithic apparatus. The fixed depth trephine 200 disclosed herein includes a cutting blade or trephine 201 mounted inside an inner reticle 203, an outer housing 202 having a substantially cylindrical shape and an outer diameter, and an inner reticle 203 having a substantially cylindrical shape and an inner diameter. These parts are configured to be manufactured as a single, monolithic piece.

The outer housing 202 has an outer, cylindrical shape although the outer shape can vary. The outer housing 202 has a distal end 205 and a proximal end 210. The proximal end 210 defines an opening with shelf 215 positioned within the outer housing 202. The shelf 215 is distally offset from the proximal end 210 along the longitudinal dimension of the outer housing 202. In this manner, there is a step formed between the proximal end 210 and the shelf 215.

The step formed between the proximal end 210 and the shelf 215 defines a depth d. The trephine 201 mounted inside the inner reticle 203 extends from the inner reticle 203 toward the proximal end 210 and the distance of the extension of the trephine 201 from the inner reticle 203 is equal to the depth d. Therefore, the partial thickness cut formed by the trephine 201 has the depth d that is equal to the depth defined by the step formed between the proximal end 210 and the shelf 215.

In some embodiments, the inner reticle 203 forms the inner diameter depth fixation shelf 215. For example, the inner diameter depth fixation shelf 215 may be configured as a single contiguous piece of the inner diameter. In other embodiments, the inner diameter depth fixation shelf 215 may be configured as a separate piece insertable in the inner reticle 203. For example, the inner depth fixation shelf 215 may be inserted into the inner reticle 203 and held in place via snap fit, friction fit, interference fit, press fit, adhesive coupling, magnetic coupling, or other joining mechanisms suitable to hold the inner depth fixation shelf 215 in place without allowing movement of the inner depth fixation shelf 215 relative to the inner reticle 203 when the fixed depth trephine 200 is in use.

The fixed depth trephine 200 may be composed of a plastic or composite material, a polymer, a metal or metal alloy, or other suitable material. The fixed depth trephine 200 may be manufactured by cast molding, injection molding, extrusion, polymer casting, 3D printing, CNC machining, or other method suitable to the material used to manufacture the fixed depth trephine 200.

The fixed depth trephine 200 may be configured for single-use, i.e. disposable, or may be reused and sterilized between uses. The fixed depth trephine 200 may be configured as a non-vacuum or a suction trephine. The fixed depth trephine 200 may be used in, but is not limited to, ophthalmic surgeries.

### I. Definitions

The term “trephine” as used herein refers to a surgical instrument with a blade, such as a cylindrical blade, to form an incision in a tissue and/or to remove a portion of a tissue in a patient undergoing the surgery.

“Patient” or “subject” as used herein refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A surgical instrument comprising:
a cutting blade;
an outer housing having an outer diameter; and
an inner housing having an inner diameter and an inner diameter shelf,
wherein the inner diameter shelf is configured as a depth fixation apparatus.

2. The surgical instrument of claim 1, wherein the cutting blade, the outer housing, the inner housing, and the inner diameter shelf are configured as a single piece.

3. The surgical instrument of claim 1, wherein the cutting blade, the outer housing, and the inner housing are configured as a first piece, and wherein the inner diameter shelf is configured as a second piece insertable into the inner housing of the first piece.

4. The surgical instrument of claim 1, wherein the surgical instrument is disposable.

5. The surgical instrument of claim 1, wherein the surgical instrument is reusable.

6. The surgical instrument of claim 1, wherein the surgical instrument is a trephine.

7. A method of forming an incision in a tissue, the method comprising:
setting a desired blade depth using a depth fixation apparatus of a surgical instrument;
placing an outer housing of the surgical instrument on a surface of the tissue to be cut; and
forming an incision in the tissue, the incision having a desired depth.

8. The method of claim 7, wherein the depth fixation apparatus of the surgical instrument comprises an inner diameter shelf.

9. The method of claim 8, wherein the inner diameter shelf forms a contiguous part of the surgical instrument.

10. The method of claim 8, wherein the inner diameter shelf comprises a separate piece that is coupled to the surgical instrument.

11. The method of claim 7, wherein the surgical instrument is a trephine.
